(19) United States
(12) Patent Application Publication
(10) Pub. No.: US 2016/0184002 A1
(43) Pub. Date: Jun. 30, 2016

(54) DEVICE FOR ENHANCING INDUCED MAGNETIC FIELD CONSEQUENT FOR THERMAL ABLATION THERAPY

(71) Applicant: METAL INDUSTRIES RESEARCH AND DEVELOPMENT CENTRE, Kaohsiung (TW)

(72) Inventors: Tung-Chieh YANG, Kaohsiung (TW); Tsung-Chih YU, Kaohsiung (TW); Yi-San CHANG, Kaohsiung (TW); Xi-Zhang LIN, Kaohsiung (TW); Syuan-Fong CHEN, Kaohsiung (TW); Guan-Cheng CHEN, Kaohsiung (TW)

(21) Appl. No.: 14/586,024

(22) Filed: Dec. 30, 2014

(51) Int. Cl. A61B 18/10; H01F 1/42; H01F 1/14; H01F 1/147; H01F 5/00; H01F 7/20

(52) U.S. Cl. CPC A61B 18/10; H01F 5/00; H01F 7/20; H01F 1/14; H01F 1/147; H01F 1/42; A61B 2018/00577

(57) ABSTRACT

A device for enhancing induced magnetic field consequent for thermal ablation therapy includes a magnetic field generating unit, an annular support and a magnetic ring. The annular support is non-magnetic and non-electrically-conductive, and has two annular end faces and annular outer and inner walls. The magnetic field generating unit is fixed around and contacts the annular outer wall. The magnetic ring is magnetic, has a ring body confining a ring opening, fixed to one of the annular inner wall and the annular end faces, and extending around a common axial line that extends through the annular inner wall. The ring opening is smaller than the annular inner wall in dimension on a plane perpendicular to the common axial line.

### FIELD OF THE DISCLOSURE

The disclosure relates to a device for enhancing induced magnetic field consequent for thermal ablation therapy.

### BACKGROUND OF THE DISCLOSURE

Recently, there is a new technology adapted for tumor treatment that can save costs for patients and reduce the pain inflicted by the operation. A thermal ablation therapy is to apply an alternating magnetic field on a magnetizable needle, heat the needle with energy from eddy currents generated by the alternating magnetic field, then use the heated needle to ablate a tumor.

Referring to FIG. 1, Taiwanese Patent Publication No. 201410290 (Taiwanese Patent Application No. 101133801) discloses a deep magnetic field generating apparatus 1 which includes a first coil unit 11 and a second coil unit 12 that is parallel to, surrounds and is connected to the first coil unit 11. The first coil unit 11 can generate a concentrated alternating magnetic field by electrically connecting to an alternating electric power source, and the second coil unit 12 can generate a large-area alternating magnetic field and cooperate with the first coil unit 11 to generate an alternating magnetic field, which can reach the deep inside a human body. Further referring to FIG. 2, the alternating magnetic field can reach a magnetizable needle 2 inserted inside of a human body 3, and provide heat energy to ablate a tumor 31 inside of the human body 3 without an electrical contact to the human body 3.

However, the alternating magnetic field generated by the deep magnetic field generating apparatus 1 may diffuse before reaching the magnetizable needle 2, which may affect production of heat energy to the magnetizable needle 2 for the ablation of the tumor 31.

### SUMMARY OF THE DISCLOSURE

Therefore, the object of the present disclosure is to provide a device for enhancing induced magnetic field consequent for thermal ablation therapy that can induce a focusing of the alternating magnetic field.

Accordingly, a device for enhancing induced magnetic field consequent for thermal ablation therapy includes a magnetic field generating unit, an annular support, and a magnetic ring. The magnetic field generating unit has a coiled portion and two connection portions. The coiled portion has two ends that are respectively connected to the connection portions which are connectible to an alternating electric power source to generate a magnetic field. The annular support is made of a non-magnetic and non-electrically-conductive material, and has an annular outer wall, an annular inner wall that is formed within the annular outer wall, a first annular end face, and a second annular end face. Each of the annular outer and inner walls is connected between the first and second annular end faces. The coiled portion of the magnetic field generating unit is fixed around and contacts the annular outer wall. The magnetic ring is made of a magnetic material and has a ring body that confines a ring opening. The ring body is fixed to one of the annular inner wall, and the first and second annular end faces. The ring body extends around a common axial line that extends through the annular inner wall. The ring opening is smaller than the annular inner wall in dimension on a plane perpendicular to the common axial line.

### BRIEF DESCRIPTION OF THE DRAWINGS

Other features and advantages of the present disclosure will become apparent in the following detailed description of the embodiments with reference to the accompanying drawings, of which:

FIG. 1 is a perspective view illustrating a deep magnetic field generating apparatus;

FIG. 2 is a perspective view illustrating an operation of the deep magnetic field generating apparatus;

FIG. 3 is an exploded perspective view illustrating a first embodiment of a device for enhancing induced magnetic field consequent for thermal ablation therapy according to the disclosure;

FIG. 4 is a sectional view of the first embodiment;

FIG. 5 is an assembled perspective view illustrating an operation of the first embodiment;

FIG. 6 is a perspective view illustrating a second embodiment of the device for enhancing induced magnetic field consequent for thermal ablation therapy according to the disclosure;

FIG. 7 is a perspective view illustrating a third embodiment of the device for enhancing induced magnetic field consequent for thermal ablation therapy according to the disclosure;

FIG. 8 is a perspective view illustrating an operation of the third embodiment; and

FIG. 9 is a sectional view illustrating a fourth embodiment of the device for enhancing induced magnetic field consequent for thermal ablation therapy according to the disclosure.

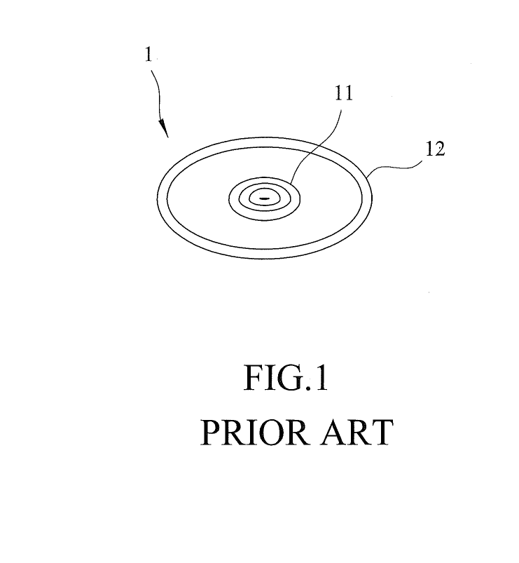

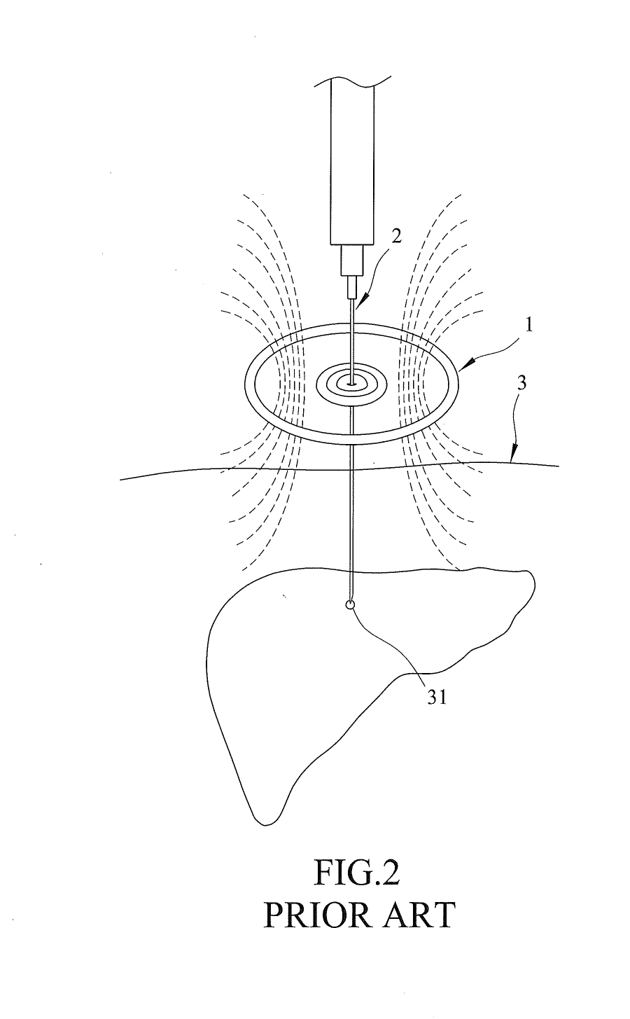

### DETAILED DESCRIPTION OF THE EMBODIMENTS

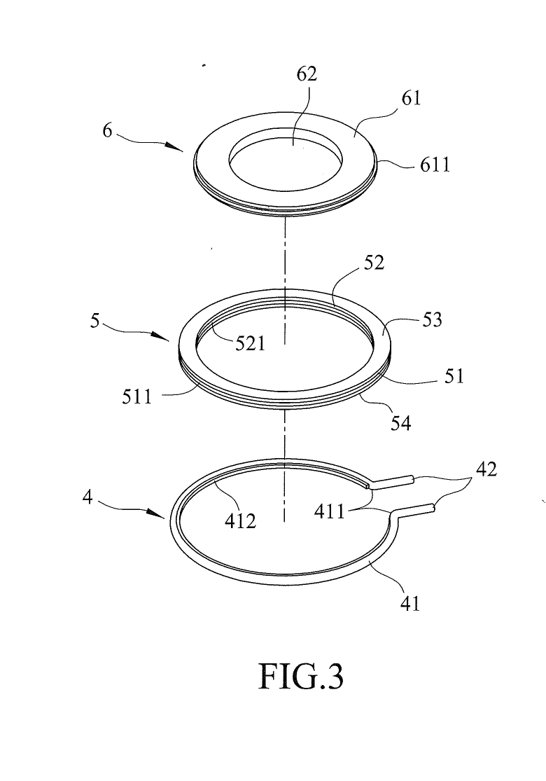

Referring to FIG. 3, a first embodiment of a device for enhancing induced magnetic field consequent for thermal ablation therapy according to the disclosure includes a magnetic field generating unit 4, an annular support 5, and a magnetic ring 6.

The magnetic field generating unit 4 has a coiled portion 41 which is made of an electrically-conductive material and formed in C-shape, and two connection portions 42. The coiled portion 41 has two ends 411 that are respectively connected to the connection portions 42 which are connectible to an alternating electric power source (not shown) to generate a magnetic field, more specifically an alternating magnetic field due to the alternating currents from the alternating electric power source. It should be noted that the shape of the coiled portion 41 is not limited by this embodiment, and may also be formed to be U-shaped, rectangular or any other shape as long as a stream of the magnetic field can be formed.

The annular support 5 is made of a non-magnetic and non-electrically-conductive material. The annular support 5 has an annular outer wall 51, an annular inner wall 52 that is formed within the annular outer wall 51, a first annular end face 53, and a second annular end face 54. Each of the annular outer and inner walls 51, 52 are connected between the first and second annular end faces 53, 54.

The magnetic ring 6 is made of a magnetic material, which may preferably be selected from an iron, an iron oxide and a carbon steel. The magnetic ring 6 has a ring body 61 that confines a ring opening 62. The ring body 61 extends around a common axial line (L) that extends through the annular inner wall 52. The ring opening 62 is smaller than the annular inner wall 52 in dimension on a plane perpendicular to the common axial line (L).

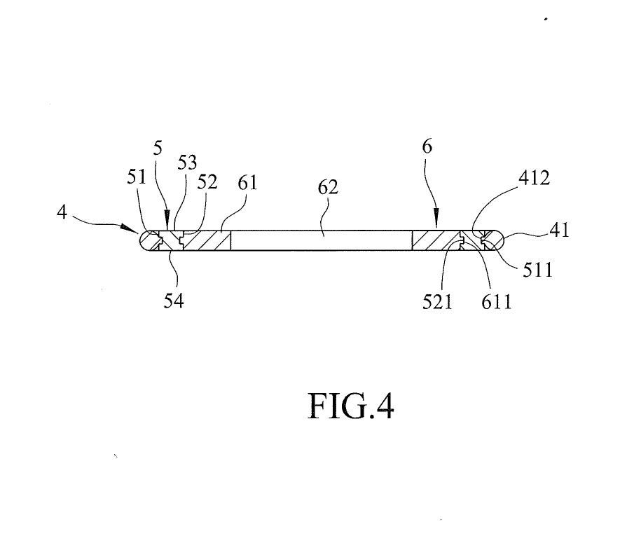

The coiled portion 412 is fixed fittingly around and contacts the annular outer wall 51. Referring to FIGS. 3 and 4, a structural configuration between the coiled portion 412 of the magnetic field generating unit 4 and the annular outer wall 51 is exemplified. In this embodiment, the coiled portion 41 is formed with an annular inner rib 41, and the annular outer wall 51 of the annular support 5 is formed with an annular outer groove 511 interlocking with the annular inner rib 41, such that the coiled portion 41 and the annular outer wall 51 are fitted complementarily to each other. Notice that, the relationship between the coiled portion 41 and the annular outer wall 51 is not limited by this embodiment. For example, the coiled portion 41 may be formed with an annular groove, and the annular outer wall 51 may be, formed with an annular rib interlocking with the annular groove.

The annular inner wall 52 of the annular support 5 is fixed fittingly around and contacts the ring body 61 of the magnetic ring 6. In this embodiment, the annular inner wall 52 is formed with an annular inner groove 521, and the ring body 61 is formed with an annular outer rib 611 interlocking with the annular inner groove 521. It should be noted that the relationship between the annular inner wall 52 and the ring body 61 is not limited by this embodiment. For example, the ring body 61 may be formed with an annular groove, and the annular inner wall 52 may be formed with an annular rib interlocking with the annular groove.

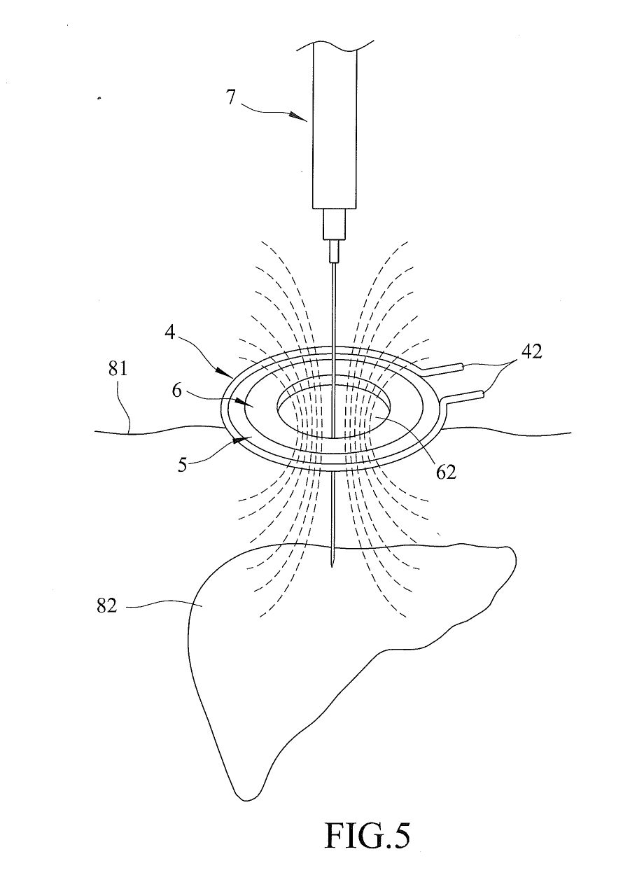

Referring to FIG. 5, the device for enhancing induced magnetic field consequent for thermal ablation therapy can cooperate with an auxiliary treatment tool 7, which is a magnetizable needle in this embodiment, to conduct a tumor treatment. In use, the connection portions 42 of the magnetic field generating unit 4 are first connected to the alternating electric power source to generate an alternating magnetic field from the magnetic field generating unit 4, and the device for enhancing induced magnetic field consequent for thermal ablation therapy is moved to be close to an outer skin 81 of a human body. Then, due to the affection of the magnetic ring 6, the alternating magnetic field is drawn to be directed toward a tumor 82 inside of a human body. The auxiliary treatment tool 7 is then passed through the ring opening 62 of the magnetic ring 6 and penetrates the outer skin 81 to the tumor 82 inside of the human body. By virtue of the alternating magnetic field nearby the tumor 82, eddy currents are created at an end of the auxiliary treatment tool 7 nearby the tumor 82, thereby producing heat energy to ablate the tumor 82.

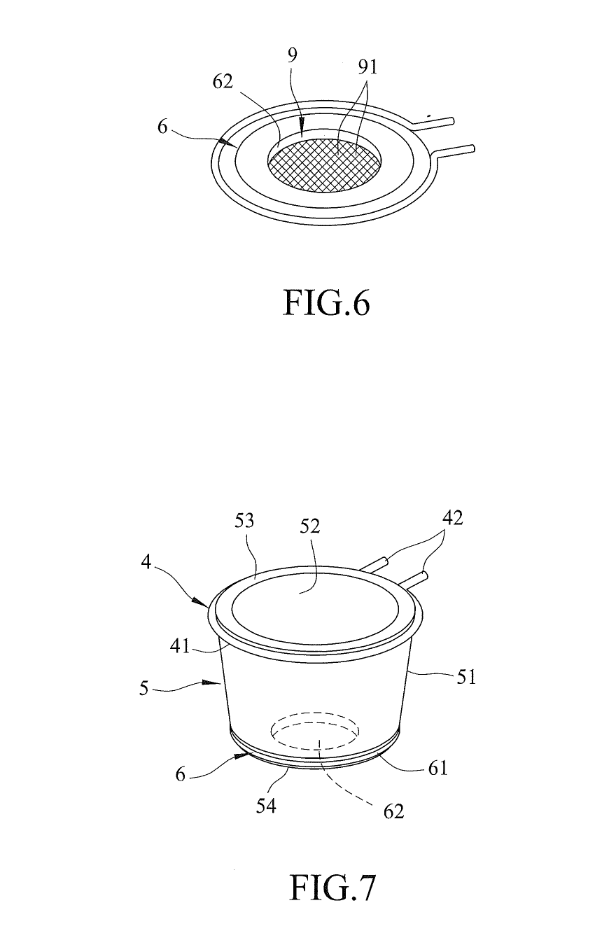

Referring to FIG. 6, a second embodiment of the device for enhancing induced magnetic field consequent for thermal ablation therapy according to the disclosure further includes a positioning member 9 which is disposed in the ring opening 62 of the magnetic ring 6 and has a plurality of screen holes 91. The positioning member 9 is made of a magnetic material, which may preferably be selected from an iron, an iron oxide and a carbon steel. The presence of the screen holes 91 would facilitate precise penetration of the auxiliary treatment tool 7 (see FIG. 5) in the tumor treatment. Also, the screen holes 91 can aid the concentration of the alternating magnetic field generated by the magnetic field generating unit 4 so as to increase tumor treatment efficiency.

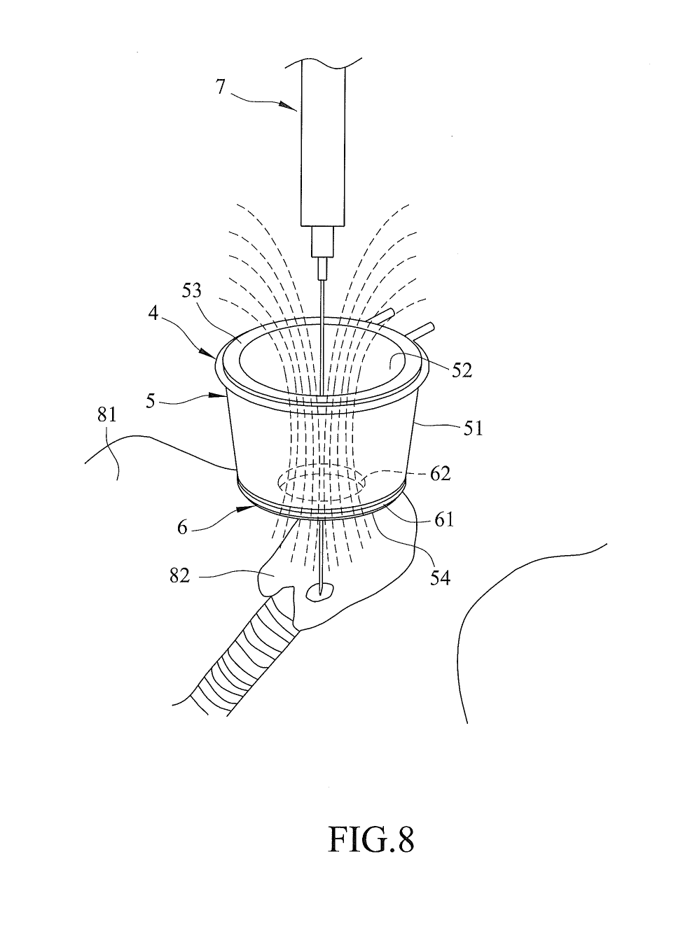

Referring to FIGS. 7 and 8, a third embodiment of the device for enhancing induced magnetic field consequent for thermal ablation therapy according to the disclosure has a structure similar to that of the first embodiment. The difference between the third embodiment and the first embodiment resides in that the annular support 5 is formed in tubular shape. Each of the annular outer and inner walls 51, 52 tapers from the first annular end face 53 to the second annular end face 54. The coiled portion 41 of the magnetic field generating unit 4 is fixed around and contacts the annular outer wall 51 at a position adjacent to the first annular end face 53, and the ring body 61 of the magnetic ring 6 is fixed to the second annular end face 54. As such, the device for enhancing induced magnetic field consequent for thermal ablation therapy is suitable for use on finer parts of the human body, such as the neck, since the magnetic ring 6 can be placed much closer to the tumor 82. In addition, the tubular annular support 5 further concentrates the alternating magnetic field and increases tumor treatment efficiency.

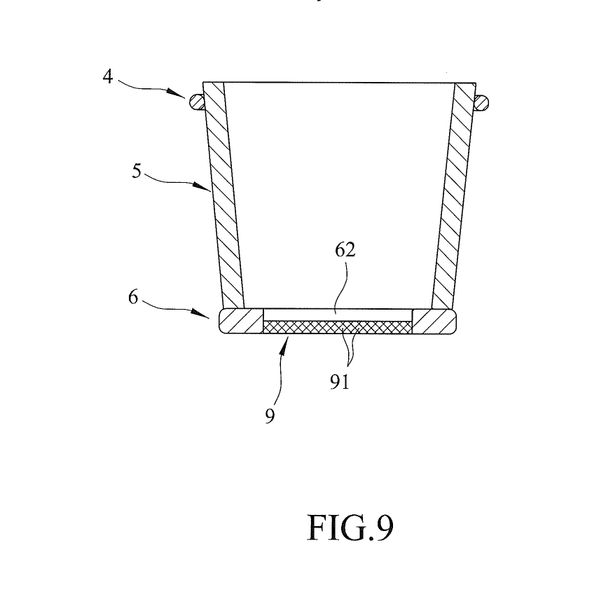

Referring to FIG. 9, a fourth embodiment of the device for enhancing induced magnetic field consequent for thermal ablation therapy according to the disclosure has a structure similar to that of the third embodiment. The difference between the fourth embodiment and the third embodiment resides in that the device for enhancing induced magnetic field consequent for thermal ablation therapy further includes a positioning member 9 which is disposed in the ring opening 62 of the magnetic ring 6 and has a plurality of screen holes 91. The positioning member 9 is made of a magnetic material, which may preferably be selected from an iron, an iron oxide and a carbon steel. The presence of the screen holes 91 would facilitate precise penetration of the auxiliary treatment tool 7 (see FIG. 5) in the tumor treatment.

To summarize the description above, by virtue of the magnetic ring 6, the device for enhancing induced magnetic field consequent for thermal ablation therapy according to the disclosure can improve the concentration of the alternating magnetic field generated by the magnetic field generating unit 4 to increase the efficiency of producing heat energy to the auxiliary treatment tool 7 for ablating the tumor 82 inside of the human body. Furthermore, the presence of the screen holes 91 would facilitate precise penetration of the auxiliary treatment tool 7 (see FIG. 5) in the tumor treatment. Moreover, by virtue of the tapering tubular shape of the annular support 5, the device for enhancing induced magnetic field consequent for thermal ablation therapy can be used on finer parts of the human body, and the tubular annular support 5 can further concentrate the alternating magnetic field so that tumor treatment efficiency is increased.

While the present invention has been described in connection with what are considered the most practical embodiments, it is understood that this invention is not limited to the disclosed embodiments but is intended to cover various arrangements included within the spirit and scope of the broadest interpretation so as to encompass all such modifications and equivalent arrangements.

What is claimed is:

1. A device for enhancing induced magnetic field consequent for thermal ablation therapy, comprising:
a magnetic field generating unit having a coiled portion and two connection portions, said coiled portion having two ends that are respectively connected to said connection portions which are connectible to an alternating electric power source to generate a magnetic field;
an annular support made of a non-magnetic and non-electrically-conductive material, and having an annular outer wall, an annular inner wall that is formed within said annular outer wall, a first annular end face, and a second annular end face, each of said annular outer and inner walls being connected between said first and second annular end faces, said coiled portion of said magnetic field generating unit being fixed around and contacting said annular outer wall; and
a magnetic ring made of a magnetic material and having a ring body that confines a ring opening, said ring body being fixed to one of said annular inner wall, and said first and second annular end faces, said ring body extending around a common axial line that extends through said annular inner wall, said ring opening being smaller than said annular inner wall in dimension on a plane that is perpendicular to the common axial line.

2. The device for enhancing induced magnetic field consequent for thermal ablation therapy as claimed in claim 1, wherein said annular outer wall and said coiled portion are fitted complementarily to each other.

3. The device for enhancing induced magnetic field consequent for thermal ablation therapy as claimed in claim 1, wherein:
one of said annular outer wall and said coiled portion is formed with an annular groove; and
the other one of said annular outer wall and said coiled portion is formed with an annular rib interlocking with said annular groove.

4. The device for enhancing induced magnetic field consequent for thermal ablation therapy as claimed in claim 3, wherein:
one of said annular inner wall and said ring body is formed with an annular groove; and
the other one of said annular inner wall and said ring body is formed with an annular rib interlocking with said annular groove.

5. The device for enhancing induced magnetic field consequent for thermal ablation therapy as claimed in claim 1, further comprising a positioning member made of a magnetic material, disposed in said ring opening, and having a plurality of screen holes.

6. The device for enhancing induced magnetic field consequent for thermal ablation therapy as claimed in claim 5, wherein said positioning member is made of a material selected from an iron, an iron oxide and a carbon steel.

7. The device for enhancing induced magnetic field consequent for thermal ablation therapy as claimed in claim 1, wherein:
said annular support is tubular;
said coiled portion is fixed around and contacts said annular outer wall at a position adjacent to said first annular end face; and
said ring body is fixed to said second annular end face.

8. The device for enhancing induced magnetic field consequent for thermal ablation therapy as claimed in claim 7, wherein said each of said annular outer and inner walls tapers from said first annular end face to said second annular end face.

9. The device for enhancing induced magnetic field consequent for thermal ablation therapy as claimed in claim 7, further comprising a positioning member made of a magnetic material, disposed in said ring opening, and having a plurality of screen holes.

10. The device for enhancing induced magnetic field consequent for thermal ablation therapy as claimed in claim 9, wherein said positioning member is made of a material selected from an iron, an iron oxide and a carbon steel.

11. The device for enhancing induced magnetic field consequent for thermal ablation therapy as claimed in claim 1, wherein said magnetic ring is made of a material selected from an iron, an iron oxide and a carbon steel.
